(19) United States
(12) Patent Application Publication
(10) Pub. No.: US 2013/0261624 A1
(43) Pub. Date: Oct. 3, 2013

(54) MAXILLARY IMPLANT FOR ADVANCEMENT, EXPANSION AND STABILIZATION OF REPOSITIONED BONE SECTIONS

(71) Applicants: DALE STRINGER, Riverside, CA (US); Geronimo Hernandez, Dallas, TX (US)

(72) Inventors: DALE STRINGER, Riverside, CA (US); Geronimo Hernandez, Dallas, TX (US)

(73) Assignee: OsteoMed LLC, Addison, TX (US)

(21) Appl. No.: 13/436,213

(22) Filed: Mar. 30, 2012

(51) Int. Cl. A61B 17/80

(52) U.S. Cl. 606/71

(57) ABSTRACT

Embodiments of the invention are directed to a maxillary implant for advancement, expansion and stabilization of repositioned bone sections of a maxilla. According to embodiments, a maxillary implant includes a first plate having a bone attachment portion. The bone attachment portion has a bone contacting surface defining a first plane and one or more screw holes. The first plate has a coupling portion defining a second plane and has a post-opening. The maxillary implant includes a second plate having a top panel and two side panels. The two side panels each has a bone contacting surface defining respective third and fourth planes and one or more screw holes. The maxillary implant has a post projecting from the top panel in the posterior direction. The post and the post-opening jointly form an interface which allows the first and second plates to be adjustable relative to each other.

### TECHNICAL FIELD

The invention generally relates to orthopedic implants, and more particularly to a maxillary implant for advancement, expansion and stabilization of repositioned bone sections.

### BACKGROUND

Patients with an underdeveloped maxilla and other cranio-facial defects often undergo surgeries to reposition bone sections to treat these defects. FIG. 1 illustrates a human skull 100 including a maxilla 104. In FIG. 1, the x-axis indicates a lateral direction in relation to the maxilla 104, the y-axis indicates a superior-inferior direction in relation to the maxilla 104, and the z-axis indicates an anterior-posterior direction in relation to the maxilla 104. A patient with a transversely deficient maxilla, for example, may require maxillary advancement and expansion. One existing technique involves a two-stage surgery. In the first stage (also known as SARPE, which stands for Surgically Assisted Rapid Palatial Expansion), the maxilla is mid-palatally sectioned and the bone segments are expanded laterally. In a second stage, a LeFort osteotomy is performed to reposition the bone segments to a desired position (e.g., inferior-superior, anterior-posterior). It will be appreciated that an osteotomy is a surgical procedure whereby a bone is cut to shorten, lengthen or change its alignment. The LeFort osteotomy is a widely used surgical procedure whereby bone sections of a maxilla are repositioned. This two-staged procedure is used when the maxilla has a transverse discrepancy of greater than 5 mm and needs to be repositioned into a second position (superior, anterior or posterior). It will be appreciated by those skilled in the art that expanding the maxilla more than 5 mm by a LeFort I three-piece osteotomy alone may create a high risk of relapse.

There are disadvantages associated with existing procedures (e.g., two-stage surgery) to reposition a maxilla. Since these techniques require two equally invasive surgical procedures, the second stage surgery is often difficult to perform due to formation of scar tissue from the first stage surgery.

Various orthopedic implants, such as, for example, bone distraction devices are known in the art for stabilization of a repositioned maxilla. U.S. Pat. No. 6,908,469 discloses a maxillary distractor comprising first and second plates and an actuator attached to the plates. The actuator includes a screw for adjusting the position of the plates. U.S. Pat. No. 5,902,304 discloses a telescopic bone plate for distracting ostetomically separated bone sections. The telescopic bone plate includes top and bottom bone plates and a telescopic member for adjusting the relative position of the top and bottom plates. Since these bone distraction devices are generally used to facilitate the two-stage procedure, the risk of a loss of expansion and a formation of scar tissue remains.

### SUMMARY

Embodiments of the invention are directed to a maxillary implant for advancement, expansion and stabilization of repositioned bone sections of a maxilla. According to embodiments, a maxillary implant includes a first plate having a bone attachment portion. The bone attachment portion has a bone contacting surface defining a first plane and one or more screw holes. The first plate has a coupling portion defining a second plane and has a post-opening. The first plane may be oriented at a predetermined angle relative to the second plane. The maxillary implant includes a second plate having a top panel and two side panels. The two side panels each has a bone contacting surface defining respective third and fourth planes and one or more screw holes. The maxillary implant may have a post projecting from the top panel in the posterior direction. The post is oriented at a predetermined angle with respect to the top panel and is configured to be inserted through the post-opening. The post and the post-opening jointly form an interface which allows the first and second plates to be adjustable relative to each other in at least the anterior-posterior direction.

According to other embodiments, a maxillary implant has a first plate having a bone attachment portion. The bone attachment portion has a bone contacting surface defining a first plane and has one or more screw holes. The first plate has a coupling portion defining a second plane. The first plane may be oriented at a predetermined angle relative to the second plane. The maxillary implant has a second plate having a top panel and two side panels. The two side panels have bone contacting surfaces defining respective third and fourth planes and have one or more screw holes. The maxillary implant has an interface for allowing the first and second plates to be adjustable relative to each other in at least the anterior-posterior direction. The interface includes a post-opening in the coupling portion of the first plate and a post projecting from the top panel of the second plate.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

FIG. 1 illustrates a human skull including the maxilla;

FIGS. 2A-2D illustrate several views of a first plate of a maxillary implant according to embodiments of the invention;

FIGS. 3A and 3B illustrate a front view and a top view, respectively, of a second plate of a maxillary implant according to embodiments of the invention;

FIG. 4 illustrates a maxillary implant according to embodiments of the invention;

FIGS. 5A and 5B illustrate a maxillary implant according to other embodiments of the invention;

FIGS. 6A and 6B illustrate a front view and a top view, respectively, of a maxillary implant according to other embodiments of the invention;

FIGS. 7A and 7B illustrate a front view and a side view, respectively, of a first plate in accordance with other embodiments of the invention;

FIGS. 8A and 8B illustrate a front view and a side view, respectively, of a first plate according to other embodiments of the invention; and

FIGS. 9A and 9B illustrate maxillary implants used to stabilize repositioned bone sections of a maxilla.

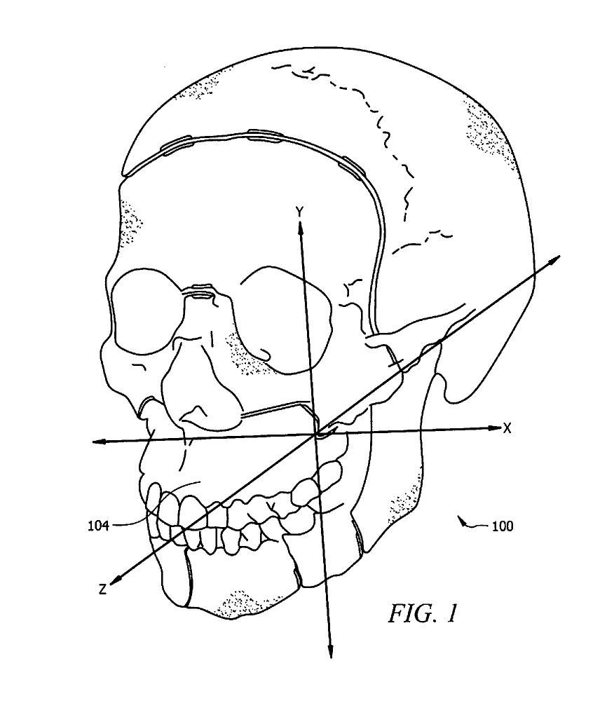

### DETAILED DESCRIPTION OF THE INVENTION

The detailed description set forth below, in connection with the appended drawings, is intended as a description of various configurations and is not intended to represent the only configurations in which the concepts described herein may be practiced. The detailed description includes specific details for the purpose of providing a thorough understanding of the various concepts. However, it will be apparent to those skilled in the art that these concepts may be practiced without these specific details. In some instances, well-known structures and components are shown in block diagram form in order to avoid obscuring such concepts.

The word “exemplary” is used herein to mean “serving as an example, instance, or illustration.” Any embodiment or aspect described herein as “exemplary” is not necessarily to be construed as preferred or advantageous over other embodiments or aspects. In addition, references to “an,” “one,” “other,” or “various” embodiments or aspects should not be construed as limiting since various aspects of the disclosed embodiments may be used interchangeably within other embodiments.

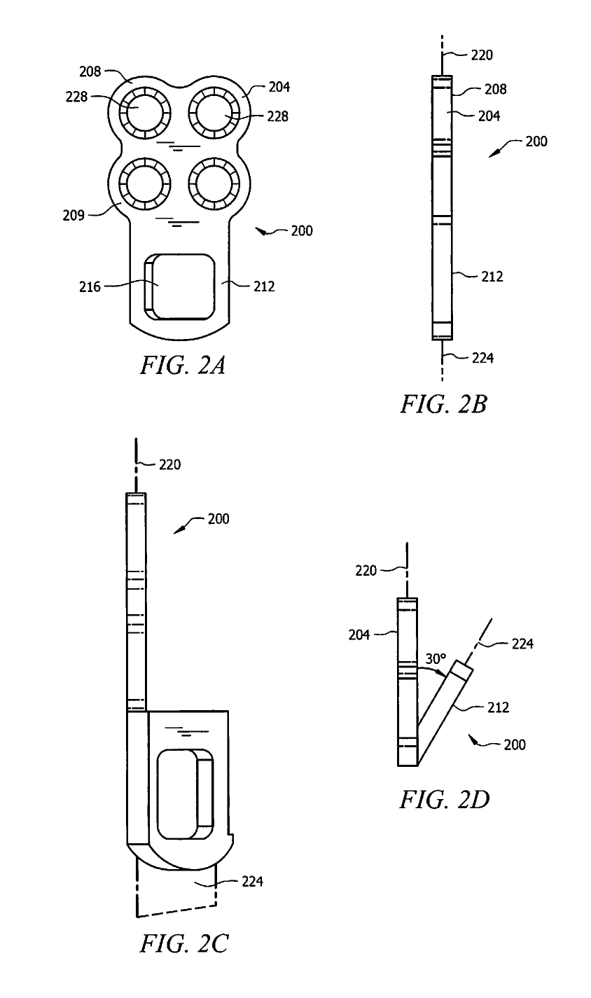

Embodiments of the invention are directed to a maxillary implant for advancement, expansion and stabilization of repositioned bone sections of a maxilla. Referring to FIG. 2A, a first plate 200 of the maxillary implant is shown according to embodiments of the invention. The shape of the first plate 200 may be adjusted as needed and does not limit the scope of the invention. The first plate 200 includes a bone attachment portion 204 having a bone contacting surface 208 opposite of a side 209. The first plate 200 includes a coupling portion 212 adjacent to the bone attachment portion 204. An opening 216 is formed in the coupling portion 212. The opening 216 receives a post (not shown in FIG. 2A) projecting from a second plate. As will be explained later, the post and the opening 216 form an interface which allows the first plate 200 to be supported by the second plate. According to embodiments, the opening 216 may be circular, square or any other suitable shape for receiving a post. The first plate 200 may be made from stainless steel, titanium, metallic alloy or other material suitable for an orthopedic implant device.

Referring now to FIG. 2B, which shows a side view of the first plate 200, the bone contacting portion 204 of the first plate 200 defines a first plane 220 and the coupling portion 212 defines a second plane 224. The first and second planes 220 and 224 may be co-planar as shown in FIG. 2B or they may be oriented at a predetermined angle with respect to one another. FIG. 2C illustrates a first plate 200 according to embodiments of the invention wherein the first plane 220 is at an angle relative to the second plane 224. FIG. 2D illustrates a top view of the plate 200 showing a 30 degree angle between the first plane 220 and the second plane 224. It will be appreciated that the angular orientation of the first plane 220 relative to the second plane 224 may vary. Also, it will be apparent that multiple planes in the plate 200 may be obtained by bending the plate 200.

Referring still to FIG. 2A, the bone attachment portion 204 includes one or more screw holes 228 adapted to receive attachment screws to fixate the first plate 200 to a bone section. For example, the first plate 200 may be fixated to an osteotomically separated and repositioned bone section of a maxilla by one or more attachment screws through the screw holes 228.

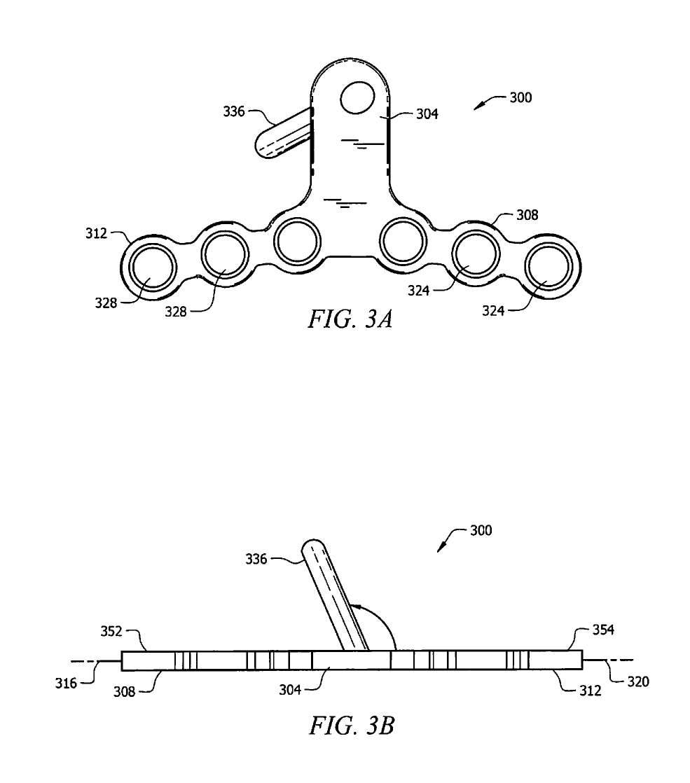

FIG. 3A illustrates a second plate 300 according to embodiments of the invention. The shape of the second plate 300 may be adjusted as needed and does not limit the scope of the invention. The second plate 300 includes a top panel 304 and two side panels 308 and 312. FIG. 3B illustrates a top view of the second plate 300. The two side panels 308 and 312 have bone contacting surfaces 352 and 354 and define third and fourth planes 316 and 320, respectively. The planes 316 and 320 may be co-planar or they may be oriented at a predetermined angle with respect to one another. The side panels 308 and 312 each have one or more screw holes 324 and 328 adapted to receive attachment screws (not shown) to fixate the second plate 300 to a bone section. The second plate 300 may, for example, be fixated by attachment screws to an osteotomically separated and repositioned bone section of a maxilla.

According to embodiments, the second plate 300 includes at least one post 336 projecting from the top panel 304. The post 336 may be oriented at a predetermined angle with respect to the top panel 304. By way of example, the post 336 may be oriented at a predetermined angle with respect to the top panel 304 or may be substantially perpendicular to the top panel 304. Accordingly, the post 336 may be oriented at predetermined angles with respect to the planes 316 and 320 or may be substantially perpendicular to the planes 316 and 320. The post 336 is configured to be inserted through the opening 216 formed in the first plate 200. As noted before, the post 336 and the post opening 216 form an interface which allows the first plate 200 to support the second plate 300. The interface also allows the first and second plates 200 and 300 to adjust relative to one another in at least the anterior-posterior direction. The second plate 300 may be made from stainless steel, titanium, metallic alloy or other material suitable for an orthopedic implant device.

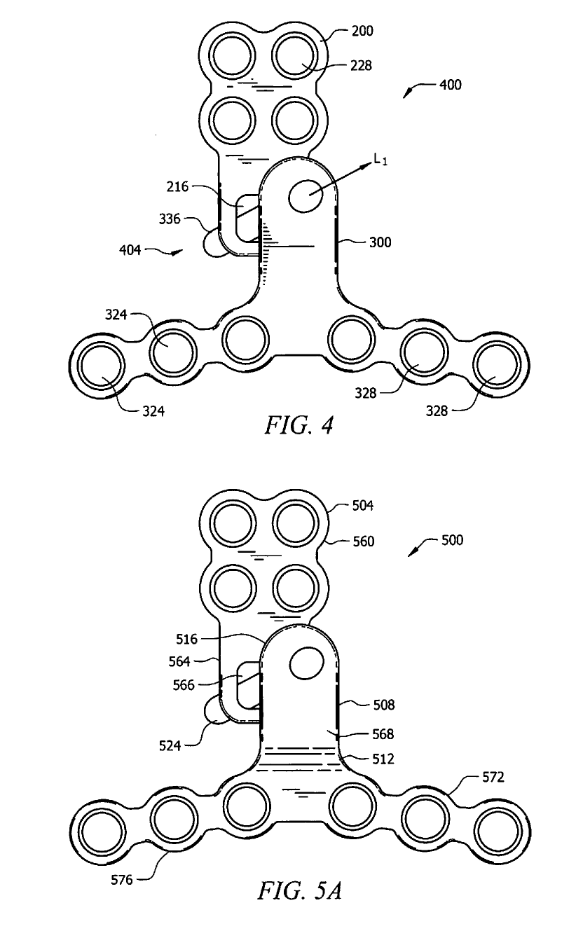

FIG. 4 illustrates a maxillary implant 400 showing the first plate 200 and the second plate 300. An interface 404, which comprises the post 336 projecting from the second plate 300 and the opening 216 formed in the first plate 200, allows the first and second plates 200 and 300 to adjust relative to one another in at least the anterior-posterior direction, and possibly in the inferior-superior direction, indicated by a line L1. The line L1 may be oriented at a predetermined angle (e.g., 30 degrees, 60 degrees, etc.) with respect to the opening 216. The interface 404 also allows the first plate 200 to rotate about the second plate 300. It will be apparent that multiple planes in the plates 200 and 300 may be obtained by bending the plates 200 and 300. The screw holes 228 formed in the first plate 200 receives attachment screws (not shown in FIG. 4) for fixating the first plate 200 to a bone section such as, for example, a bone section of a maxilla. Likewise, the screw holes 324 and 328 formed in the second plate 300 receives attachment screws (not shown in FIG. 4) to fixate the second plate 300 to a bone section. The maxillary implant 400 provides stability to bone sections of a maxilla, for example, which may have been osteotomically separated and relocated by expansion and advancement.

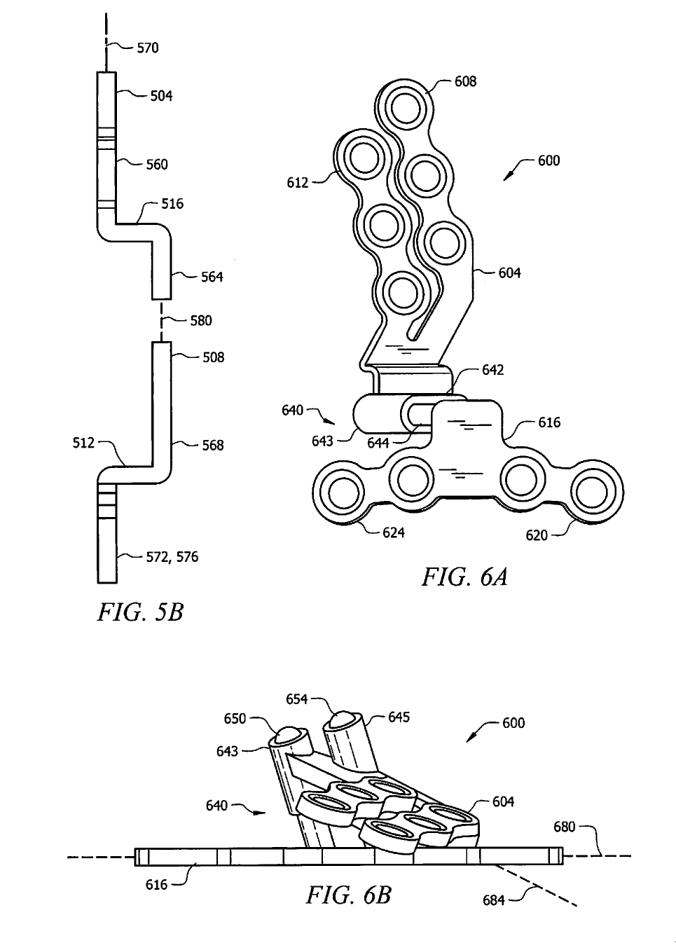

FIG. 5A illustrates a maxillary implant 500 according to other embodiments of the invention. The maxillary implant 500 comprises a first plate 504 and a second plate 508. The first plate 504 includes a bone attachment portion 560 and a coupling portion 564. An opening 566 is formed in the coupling portion 564. A step 516 is formed between the bone attachment portion 560 and the coupling portion 564.

The second plate 508 includes a top panel 568 and two side panels 572 and 576. A post 524 projects from the top panel 568. The post 524 may be oriented at an angle of approximately 60 degrees, 45 degrees or any other desired angle in the inferior-superior direction and/or lateral-medial direction in relation to the top panel 568. A step 512 is formed between the top panel 568 and the two side panels 572 and 576. It should be noted that although the first plate 504 and the second plate 508 are both shown having a step, embodiments of the invention may provide a step in only one of the plates 504 and 508. It will be apparent that multiple planes in the plates 504 and 508 may be obtained by bending the plates 504 and 508. FIG. 5B illustrates a side view of the plates 504 and 508. The step 516 is formed between the bone attachment portion 560 and the coupling portion 564 of the first plate 504. The step 512 is formed between the top panel 568 and the two side panels 572 and 576 of the second plate 508. It should be noted that the orientation and/or direction of the steps 512 and 516 may be changed according to design considerations and the needs of the patient in moving the bone portions of the maxilla being contacted by plates 504 and 508. According to embodiments, a plane 570 defined by the bone attachment portion 560 may be parallel to a plane 580 defined by the top panel. Alternatively, the planes 570 and 580 may be oriented at a predetermined angle with respect to each other.

FIG. 6A illustrates a maxillary implant 600 according to other embodiments of the invention. The maxillary implant 600 comprises a first plate 604 and a second plate 616. The first plate 604 includes a first panel 608 and a second panel 612. The second plate 616 includes two side panels 620 and 624. The shape of the first plate 604 and the second plate 616 may be adjusted as needed and does not limit the scope of the invention. The maxillary device 600 includes an interface 640 comprising a barrel 642 attached to the first plate 604. The barrel 642 includes an opening 643 sized to receive a post 644 projecting from the second plate 616. In other embodiments, the interface 640 may comprise a plurality of barrels (not shown in FIG. 6A) each configured to receive a respective post projecting from the second plate 616. It will be appreciated that the interface 640 allows free lateral motion in relation to a patient by the first and second plates 604 and 616 while significantly preventing motion in other directions.

FIG. 6B shows a top view of the maxillary implant 600 of FIG. 6A. The interface 640 includes barrels 643 and 645 having respective openings 650 and 654. The openings 650 and 654 and posts projecting from the second plate 616 may be angled so that a first plane 684 defined by the first plate 604 and a second plane 680 defined by the second plate 616 are oriented at a predetermined angle with respect to one another. It will be appreciated that by orienting the first plate 604 and the second plate 616 at a predetermined angle with respect to one another, the maxillary implant 600 may closely conform to bone sections of a maxilla (not shown in FIGS. 6A and 6B). It will be appreciated that embodiments of the invention may have interfaces that vary in configuration and design to enable free lateral motion of maxillary fragments and limited motion in all other planes by the first and second plates. Also, embodiments of the invention may have interfaces that vary in configuration and design to enable free lateral motion of maxillary fragments and to prevent motion in all other directions by the first and second plates.

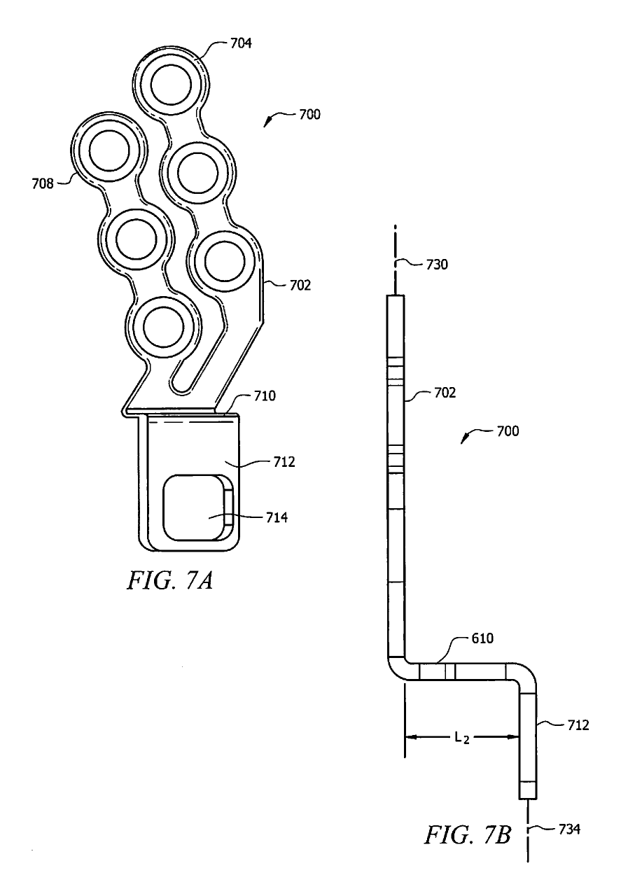

FIG. 7A illustrates a first plate 700 according to other embodiments of the invention. The first plate 700 includes a bone attachment portion 702 having first and second panels 704 and 708, respectively. The first plate 700 includes a coupling portion 712 having an opening 714. A step 710 is formed between the bone attachment portion 702 and the coupling portion 712. FIG. 7B shows a side view of the first plate 700. The step 710 has a predetermined length L2. The first plate 700 has a first plane 730 and a second plane 734 separated by a distance L defined by the length of the step 710. According to embodiments of the invention, the angular orientation between the first plane 730 and the second plane 734 may be adjusted by, for example, bending the first plate 700. It will be appreciated that the first plate 700 may be made from a malleable material allowing the plate 700 to be bent to form a desired angle between the first plane 730 and the second plane 734.

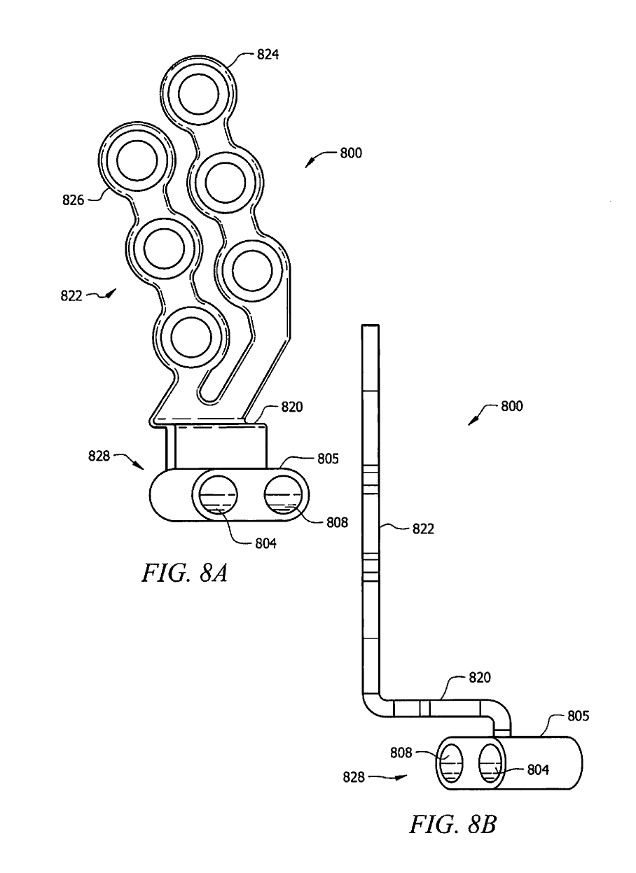

FIGS. 8A and 8B show a front view and a side view, respectively, of a first plate 800 in accordance with embodiments of the invention. The first plate 800 has a bone attachment portion 822 having first and second panels 824 and 826, respectively, and a coupling portion 828. The coupling portion 828 has a barrel 805 with openings 804 and 808 each adapted to receive a respective post from a second plate (not shown). The first plate 800 has a step 820 which connects the bone attachment portion 822 to the coupling portion 828.

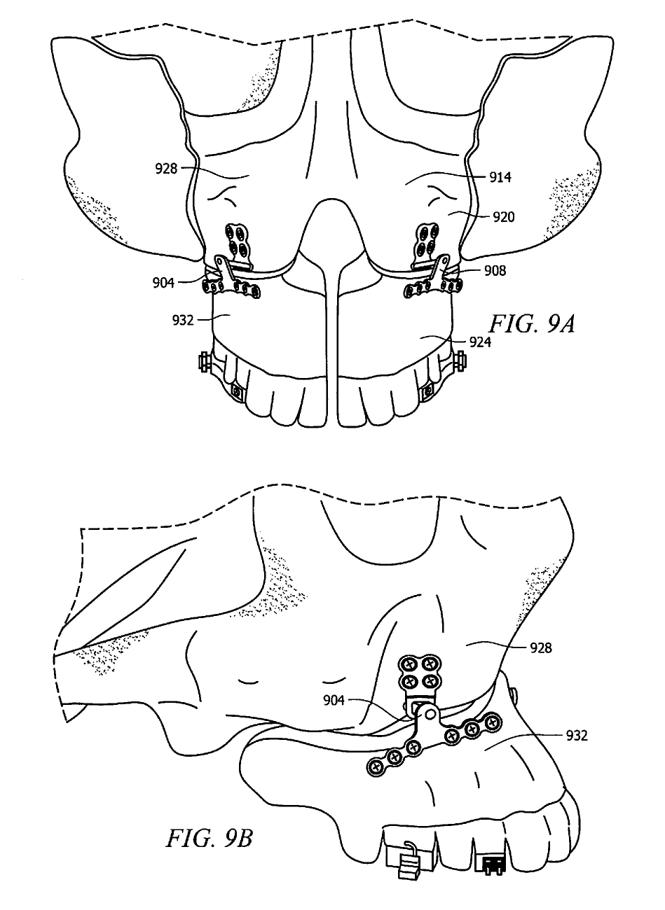

FIG. 9A illustrates two maxillary implants 904 and 908 used to stabilize osteotomically separated and repositioned bone sections of a maxilla 914. The maxilla 914 has been osteotomically separated into sections 920, 924, 928 and 932. The maxillary implant 904 is used to stabilize the repositioned sections 928 and 932, and the maxillary implant 908 is used to stabilize the repositioned sections 920 and 924. FIG. 9B illustrates a front view of the maxillary implant 904.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A maxillary implant, comprising:
a first plate having a bone attachment portion having a bone contacting surface defining a first plane, the bone attachment portion having one or more screw holes, the first plate having a coupling portion defining a second plane and having a post-opening;
a second plate having a neck and two extending arms having respective bone contacting surfaces defining respective third and fourth planes, the two arms each having one or more screw holes;
a post projecting from the neck, the post configured to be inserted through the post-opening; and
the first and second plates adjustable relative to each other in at least an anterior posterior direction.

2. The maxillary implant of claim 1, wherein the two arms extend in a substantially perpendicularly from the neck.

3. The maxillary implant of claim 1, wherein the post is substantially perpendicular to the neck.

4. The maxillary implant of claim 1, wherein the post is oriented at a predetermined angle relative to the neck.

5. The maxillary implant of claim 1, wherein the two arms are co-linear.

6. The maxillary implant of claim 1, wherein the two arms are oriented at a predetermined angle with respect to each other.

7. The maxillary implant of claim 1, wherein the neck is substantially perpendicular to the two arms.

8. The maxillary implant of claim 1, wherein the neck is oriented at a predetermined angle with respect to the two arms.

9. The maxillary implant of claim 1, wherein the third and fourth planes are co-planar.

10. The maxillary implant of claim 1, wherein the third and fourth planes are not co-planar.

11. The maxillary implant of claim 1, further comprising a step formed between the bone attachment portion and the coupling portion of the first plate.

12. The maxillary implant of claim 1, wherein the first and second planes are co-planar.

13. The maxillary implant of claim 1, wherein the first and second planes are oriented at a predetermined angle with respect to each other.

14. The maxillary implant of claim 1, wherein the first plate is configured to support the second plate.

15. The maxillary implant of claim 1, wherein the first plate is rotatable with respect to the second plate.

16. A maxillary implant, comprising:
a first plate having a bone attachment portion having a bone contacting surface defining a first plane, the bone attachment portion having one or more screw holes, the first plate having a coupling portion defining a second plane and having a post-opening, the first plane oriented at a predetermined angle relative to the second plane;
a second plate having a top panel and two side panels, the two side panels having bone contacting surfaces defining respective third and fourth planes, the two side panels each having one or more screw holes;
a post projecting from the top panel, the post oriented at a predetermined angle with respect to the top panel and configured to be inserted through the post-opening; and
the first and second plates adjustable relative to each other in at least an anterior posterior direction.

17. The maxillary implant of claim 16, wherein the first plate comprises a plurality of panels having the screw holes.

18. The maxillary implant of claim 16, further comprising a step formed between the bone attachment portion and the coupling portion.

19. The maxillary implant of claim 18, wherein the step has a predetermined length to define a distance between the first plane and the top panel.

20. The maxillary implant of claim 16, wherein the post is substantially perpendicular to the top panel.

21. The maxillary implant of claim 16, wherein the post is oriented at a predetermined angle relative to the top panel.

22. The maxillary implant of claim 16, wherein the two side panels are co-linear.

23. The maxillary implant of claim 16, wherein the two side panels are oriented at a predetermined angle with respect to each other.

24. The maxillary implant of claim 16, wherein the top panel is substantially perpendicular to the two side panels.

25. The maxillary implant of claim 16, wherein the first and second planes are co-planar.

26. A maxillary implant, comprising:
a first plate having a bone attachment portion having a bone contacting surface defining a first plane, the bone attachment portion having one or more screw holes, the first plate having a coupling portion having a plurality of post-openings;
a second plate having a top panel and two side panels, the two side panels having bone contacting surfaces defining respective second and third planes, the two side panels each having one or more screw holes;
a plurality of posts extending from the top panel, the posts oriented at a predetermined angle with respect to the top panel and configured to be inserted through the post-openings; and
the first and second plates adjustable relative to each other in at least an anterior posterior direction.

27. The maxillary implant of claim 26, wherein the first plate comprises a plurality of panels having the screw holes.

28. The maxillary implant of claim 26, further comprising a step formed between the bone attachment portion and the coupling portion.

29. The maxillary implant of claim 28, wherein the step has a predetermined length to define a distance between the top panel and the first plate.

30. The maxillary implant of claim 26, wherein the posts are substantially perpendicular to the top panel.

31. The maxillary implant of claim 26, wherein the posts are oriented at a predetermined angle relative to the top panel.

32. The maxillary implant of claim 26, wherein the posts are parallel to each other.

33. The maxillary implant of claim 26, wherein the two side panels are co-linear.

34. The maxillary implant of claim 26, wherein the two side panels are oriented at a predetermined angle with respect to each other.

35. The maxillary implant of claim 26, wherein the top panel is substantially perpendicular to the two side panels.

36. A maxillary implant, comprising:
a first plate having a bone attachment portion having a bone contacting surface defining a first plane, the bone attachment portion having one or more screw holes, the first plate having a coupling portion defining a second plane, the first plane oriented at a predetermined angle relative to the second plane;
a second plate having a top panel and two side panels, the two side panels having bone contacting surfaces defining respective third and fourth planes, the two side panels each having one or more screw holes; and
an interface for allowing the first and second plates to be adjustable relative to each other in at least an anterior posterior direction, the interface comprising a post-opening in the coupling portion of the first plate and a post projecting from the top panel of the second plate, the post-opening sized to receive the post.

37. The maxillary implant of claim 36, wherein the post is oriented at a predetermined angle with respect to the top panel.

38. The maxillary implant of claim 36, wherein the first plate comprises at least one panel having screw holes.

39. The maxillary implant of claim 36, further comprising a step formed between the bone attachment portion and the coupling portion.

40. The maxillary implant of claim 36, wherein the post is substantially perpendicular to the top panel.

41. The maxillary implant of claim 36, wherein the two side panels are co-linear.

42. The maxillary implant of claim 36, wherein the two side panels are oriented at a predetermined angle relative to each other.

43. A maxillary implant, comprising:
a first plate having a bone attachment portion having a bone contacting surface defining a first plane, the bone attachment portion having one or more screw holes, the first plate having a coupling portion defining a second plane and having a post-opening, the first plane oriented at an angle relative to the second plane, the angle between the first and second planes being adjustable by bending the first plate;
a second plate having a top panel and two side panels, the two side panels having bone contacting surfaces defining respective third and fourth planes, the two side panels each having one or more screw holes;
a post projecting from the top panel, the post oriented at a predetermined angle with respect to the top panel and configured to be inserted through the post-opening; and
the first and second plates adjustable relative to each other in at least an anterior posterior direction.

44. The maxillary implant of claim 43, wherein the first plate comprises a plurality of panels having the screw holes.

45. The maxillary implant of claim 43, further comprising a step formed between the bone attachment portion and the coupling portion.

46. The maxillary implant of claim 45, wherein the step has a predetermined length to define a distance between the first plane and the top panel.

47. The maxillary implant of claim 43, wherein the two side panels are co-linear.

48. The maxillary implant of claim 44, wherein the two side panels are oriented at a predetermined angle with respect to each other.
